(19) United States
(12) Patent Application Publication
(10) Pub. No.: US 2022/0306370 A1
(43) Pub. Date: Sep. 29, 2022

(54) CONTACT LENS CASE DRAINAGE DEVICE

(71) Applicant: Contact Caddy Inc., Clark, NJ (US)

(72) Inventor: John Michael Fela, Clark, NJ (US)

(73) Assignee: Contact Caddy Inc., Clark, NJ (US)

(21) Appl. No.: 17/703,046

(22) Filed: Mar. 24, 2022

Related U.S. Application Data
(60) Provisional application No. 63/166,410, filed on Mar. 26, 2021.

(51) Int. Cl. B65D 81/26

(52) U.S. Cl. CPC B65D 81/261; A45C 11/005

(57) ABSTRACT

A device on which to place contact lens case components to dry. The device is a mat with drainage channels formed thereon. The contact lens components are placed on spaced-apart walls that define the drainage channels and the spaced-apart wall receive the contact lens components and hold them above the drainage channels. This arrangement allows liquid solution and residue to drain from the contact lens case components place on the mat. The mat is sloped and the liquid solution flows to a lip on one end of the mat and then off of the mat. The mat has a slightly sloped base to assist in such drainage.

### CROSS REFERENCE TO RELATED APPLICATIONS

This application claims priority from and the benefit of U.S. Provisional Application No. 63/166,410, which was filed on Mar. 26, 2021, and which is incorporated by reference herein.

### TECHNICAL FIELD

Described herein is a device to properly drain contact case lens solution from a contact lens case or carrier that is received by the device in order to maintain the cleanliness and sterility of the contact lens case.

### BACKGROUND

Contact lenses are commonly used to improve the vision of a wearer and are a popular alternative to glasses. Contact lenses provide many advantages over traditional eyeglasses. Contact lenses do not break or scratch. Contact lenses do not fog up or permit moisture such as rain to collect on their surface, thereby obscuring the wearer's vision. Since eyeglasses have a frame that is supported by earpieces and a nose piece worn on the face, eyeglasses can easily be dislodged from the wearer during activity or just sudden or abrupt movements. Contact lenses, because they are carried on the eyeball and are retained thereon by surface tension, do not fall out of the wearer's eye when the wearer is engaged in even rigorous physical activity.

Contact lenses do have their own issues, however. Since contact lenses are placed on a wearer's eye, the contact lenses must be sterilized to ensure that contamination introduced into the eye is minimized or, preferably, completely absent. Contact lenses typically must be rehydrated periodically to stay moist in the wearer's eye. Such rehydrating is typically done overnight, with the contact lens left to soak in a sterile solution. However, the same contact lens case is typically used multiple time to rehydrate the contact lenses. Therefore, the contact lens case itself must also be clean and remain clean. While new contact lens cases are often supplied with the purchase of sterile contact lens solutions, there are few supplies that facilitate cleaning the contact lens case itself.

### BRIEF SUMMARY OF THE INVENTION

Described herein is a device that is a tool for cleaning the components of a contact lens case. Contact lens cases commonly have two receptacles, in a base, one receptacle for each lens. The receptacle receives the contact lens and receives sufficient sterile solution to completely cover the lens for soaking the lens therein. Each receptacle has a removable cover that is attachable to the case in any conventional manner. For example, the cover can attach to the lens receptacles via screw fit or press fit. The cover can be one structure that affixes to both receptacles, or individual covers or caps, one for each receptacle.

The device described herein has a mat surface made of a medical grade silicone, medical grade plastic, or other suitable material that does not absorb liquid. In one aspect, the device is intended to be flexible and washable. Medical grade plastics and medical grade silicones are well known and not described in detail herein. Medical grade silicones have an elasticity and durability that makes them particularly suitable as materials from which to make the device described herein.

The mat surface is configured to have multiple small channels that facilitate moisture draining away from the contact lens case base and covers when they are placed on the mat to dry. The device has an end portion that will allow the moisture that drains away from the contact lens case base and receptacle cover(s) to be channeled into an adjacent receptacle such as a sink. The mat offers the user complete control over where the moisture from the wet contact lens case base and receptacle cover(s) will drain. In one aspect, the mat can be placed adjacent to a sink and the moisture from the contact lens case base and covers will flow along the channels in the mat until it reaches (and drains into) the sink.

This device avoids allowing the sterile saline solution to drain onto bathroom counters made of materials such as granite, marble, and the like, which such saline solutions can damage. The device described herein makes it much easier for the user to drain their contact lens case components without having to place the wet contact lens case components on a “make-do” surface such as rag or paper towel, which surfaces do not facilitate draining and can permit residue to build up on the case components over time because the moisture does not completely drain away from the case components, but dries in contact with such components thereby forming a residue on the contact lens case components. Such residues can permit contamination to build up on the contact lenses themselves over time, which can irritate the eyes of the wearer.

### BRIEF DESCRIPTION OF THE DRAWINGS

The above and other objects and advantages of the invention will become readily apparent to those skilled in the art from reading the following detailed description of the invention when considered in the light of the accompanying drawings, in which:

FIG. 1 illustrates a top view of the contact case drainage device according to one aspect of the present invention.

FIG. 2 is a front perspective view of the contact case drainage device of FIG. 1.

FIG. 3 is a side view of the contact case drainage device of FIG. 1.

FIG. 4 is a perspective view of the contact case drainage device of FIG. 1

FIG. 5 is a front view of the contact case drainage device of FIG. 1.

FIG. 6 is a rear view of the contact case drainage device of FIG. 1.

FIG. 7 illustrates a bottom view of the contact case drainage device of FIG. 1.

### DETAILED DESCRIPTION

The following detailed description and appended drawings describe and illustrate various exemplary aspects of the invention. The description and drawings serve to provide one skilled in the art with sufficient guidance and information on how to make and use the invention, and are not intended to limit the scope of the invention in any manner.

Described herein is a device that is used to facilitate the drying of the components of a contact lens case while preventing any unwanted contact between the contact lens solution and sensitive counter surfaces such as marble, granite, etc. that might be damaged by such solutions. The device is a non-absorbent mat that allows saline solution and other liquid residue on the contact lens case components to drain away from the components of the contact lens case (i.e., the base and lens receptacle covers). The mat is configured such that the solution draining away from the contact lens case components is directed to an end of the mat from which the liquid may flow from the mat and into an adjacent drainage receptacle such as a sink.

In one aspect, the mat is made of non-absorbent plastic. In a further aspect, the plastic is medical grade. In a further aspect, the mat is made of a medical grade silicone that is both flexible and washable. By making the mat washable, the sterility of the mat can be preserved over time. In a further aspect, the mat has a textured draining surface with multiple drainage channels that direct the saline solution and any residue carried by the solution draining from the contact lens case components placed on the mat to drain from the mat in a target location. The target location from which such solution will drain from the mat is one end of the mat that is also configured as a low point relative to the rest of the mat. Consequently, the saline solution and residue will not build up on the mat. Nor will the saline solution and residue ultimately drain to other locations on the mat to other than the target drainage location. In this regard, the mat may have a thickness that varies over its length to ensure that the saline solution and residue are channeled to the location of the mat from which drainage is desired. For example, the mat may be supported by a pedestal that is thicker on one end than on the other, giving the pedestal a height at a first end of the mat that is greater than a height at the second end of the mat. The target location, which has a lip in one aspect, allows liquid to drain from the mat into an adjacent receptacle for the liquid. The advantages of this structure are that the saline and solution do not drain onto expensive counter surfaces (e.g., marble, stone, etc.) that can be stained or otherwise damaged by saline and saline residue.

When contact lens case components are left to dry on an absorbent surface such as a towel or paper towel, the saline may soak through the absorbent material and contact the counter surface. Use of the device described herein avoids any such contact between the wet contact lens case components and the counters on which such components are typically placed to dry when the contact lenses have been removed from the contact lens case and the contact lens case components have been rinsed to prepare them for their next use. Placing the contact lens case components on an absorbent surface to dry that does not draw the residue away from the contact lens case components can also leave debris on those components, and that debris can end up in the user's eye.

Having a dedicated device for this use avoids cross-contamination that can result if the contact lens components are placed on a surface that is used for other purposes. For example, if the contact lens components are set on a soap dish to dry, soap residue can end up on the components. Soap residue, if allowed to contact the lenses, may irritate the eye of the wearer.

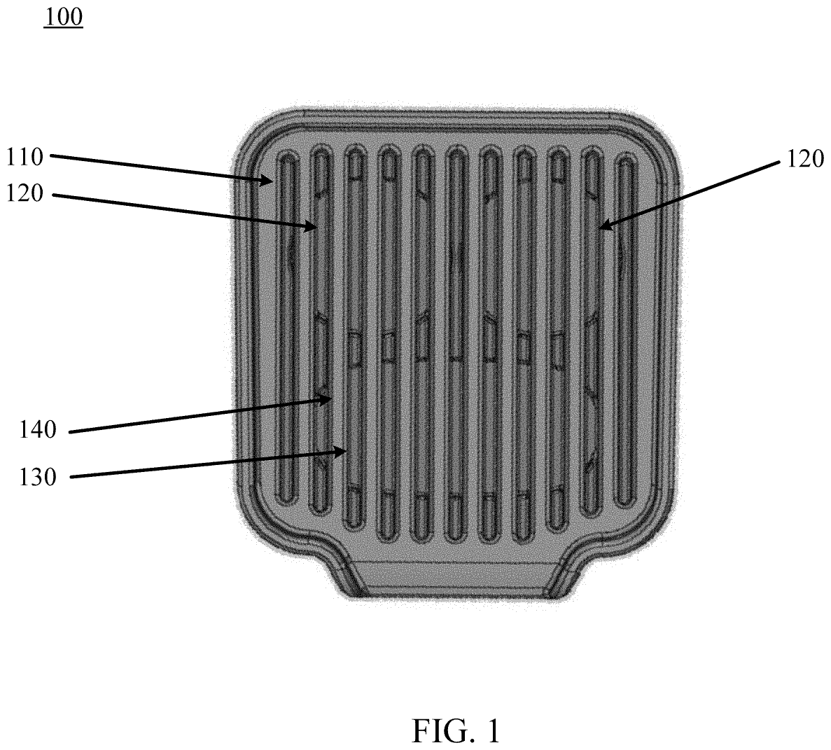

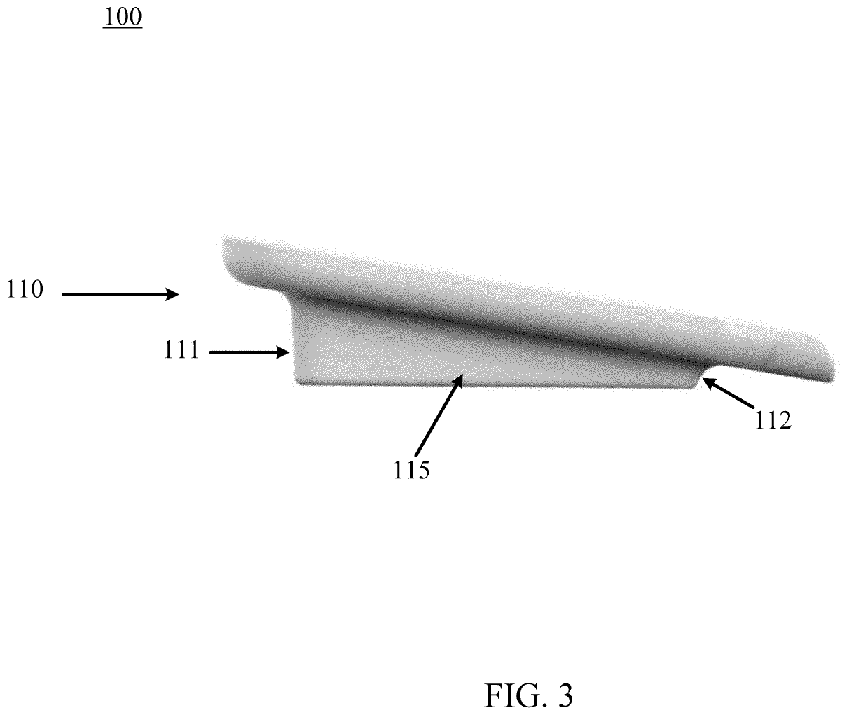

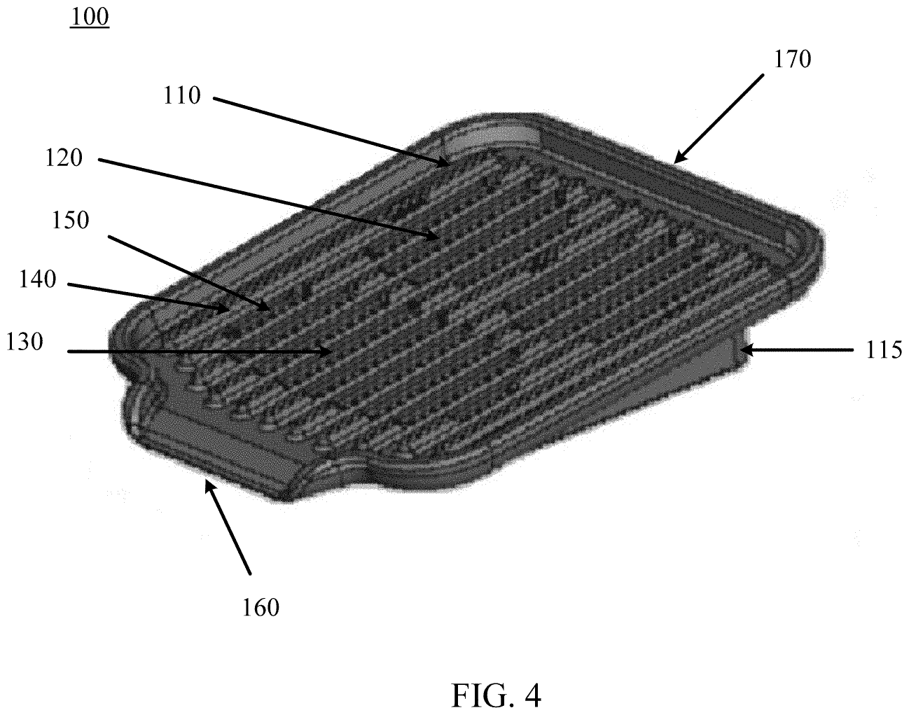

In one example, the device 100, illustrated in FIG. 1, is a mat 110 on which is configured first receptacle area 120 for receiving the contact lens case covers (not shown) thereon and second receptacle area 130 to receive the contact lens case base (also not shown). As noted above, the covers may be configured as one unitary component that fits over both contact lens receptacles in the base or two separate covers, each one fastening to one receptacle. The receptacle areas do not interrupt grooves or channels 150 (FIG. 4) that permit liquid to drain from the contact lens case components to drain away from the components and off of the mat. The dimensions of the mat 110 are largely a matter of design choice, but the dimensions should allow the mat 110 to rest entirely on a counter surface adjacent to a sink. In one aspect, mat dimensions are about 4.5 inches wide by about 5.5 inches long. As mentioned above, and with reference to FIG. 3, the mat 110 has a base 115 that has a first end with a height 111 that is greater than the height 112 of the second end, providing a slight slope (for example by varying the thickness of the mat base over its width or length to impart a slight gradation to facilitate draining the saline solution and residue from the mat and into a sink or other basin adjacent to which the mat is placed) to the mat 110. The degree of gradation is also largely a matter of design choice but a thickness variation of about one to two inches from the first end to the second end (i.e., over the width or length of the mat) should be sufficient. Thickness variations of greater or lesser amounts are contemplated. In one example, the thickness of the mat on one side is about 2.5 inches and the thickness of the mat on its other side is less than an inch. The drainage portion of the mat is flat in one aspect. FIG. 3 illustrates a mat 110 with a tapered base 115.

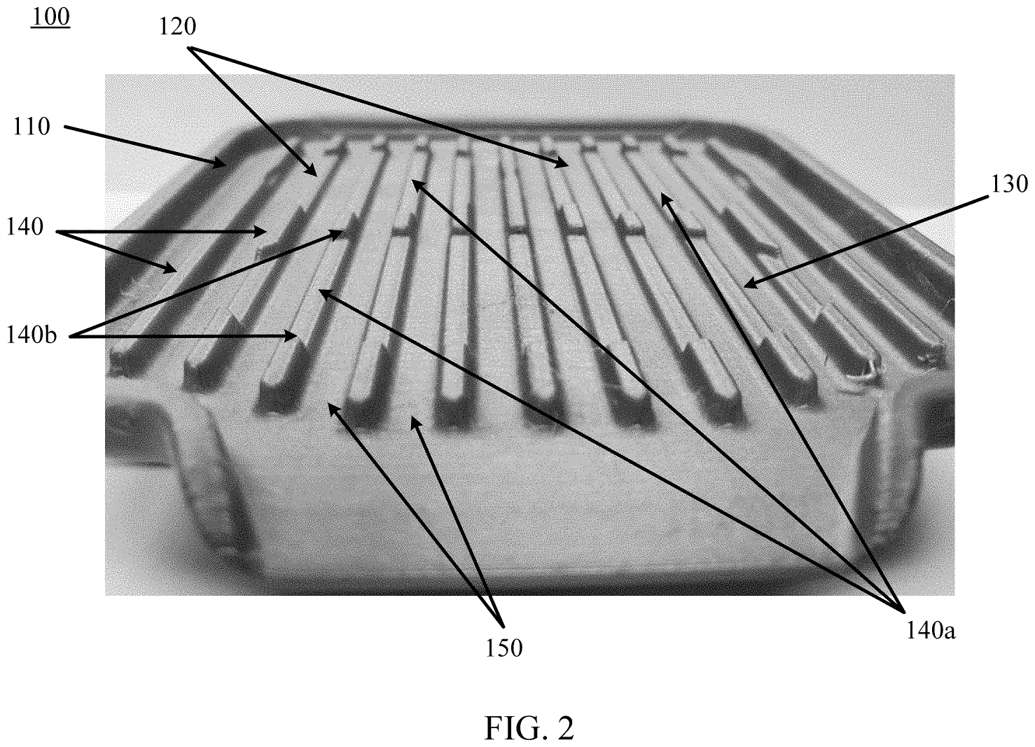

The first receptacle areas 120 for receiving the contact lens cover(s) thereon to dry provide two rounded indentations in the mat 110 in one aspect. Indentations, as used, herein, refers to the height of the walls 140 that define or separate the channels 150. The indentations are defined by a wall height 140 that is less than the wall height 140 outside first and second receptacle areas 120 and 130. The indentations are positioned to receive the contact lens covers whether the covers are separate or joined together. Similarly, the second receptacle area 130 for receiving the contact lens case base is also an indentation in the mat surface. The base of the contact lens case is received upside down so that solution and residue can drain therefrom. The first receptacle area 120 defined by the indentations are slightly larger in dimension than the contact lens case cover(s) themselves. Similarly, the second receptacle area 130 defined by the indentations and that receives the base of the contact lens case is also slightly larger than the dimensions of the contact lens case base. The first receptacle area 120 and the second receptacle area 130 in the mat 110 are illustrated in FIG. 2. As can be seen, the wall height 140a in the first and second receptacle areas is not as high as the wall height 140 outside those areas. The walls 140, 140a, define channels 150 that are uninterrupted in the first and second receptacle areas. The contact lens case base and cover(s) will fit comfortably inside the first and second receptacle areas by having, in one aspect, an additional one eighth of an inch around the perimeter of the contact lens case covers and by having about an additional one eighth of an inch around the contact lens case base, thereby providing a second receptacle area having defined by an approximately three inch by two inch area with indented wall height 140a that will receive the contact lens case base.

Since the mat surface has multiple drainage channels 150 formed thereon, the surface of the mat 110 is a number of grooves or channels with walls 140 on either side. Therefore, when the contact lens case cover(s) and base are placed on the mat 110, they are supported by the walls 140 of the channels 150 formed on the mat. As explained above, the first and second receptacle area 120, 130 are regions where the channel walls 140a have a height that is less than the height of the channel walls 140 in regions of the mat outside the first and second receptacle areas (i.e., non-receptacle regions) of the mat. The perimeter of the receptacle regions is defined by height transitions 140b. Each wall 140 traversing through the receptacle regions 120, 130 contains two height transitions from 140 to 140a, one on either side of the receptacle regions. This is illustrated in FIG. 2.

Since the top surface of the mat is preferably uniform in height in both the indented regions and the other regions of the mat, and the mat base 115 is tapered, the height of the channel walls may change as the thickness of the mat base 115 changes to ensure that the channel wall height provides an approximately constant-height top surface on which to place objects to dry. That is not to preclude mats with less consistent channel wall heights, as some non-uniformity in channel wall height will not adversely affect the ability of the mat to retain the contact lens case covers and base thereon and allow solution and residue to drain therefrom.

As noted above, the mat 110 illustrated in the figures has a grooved surface with walls 140 to provide multiple drainage channels 150, which are illustrated in FIG. 2. The channels 150 illustrated in FIG. 2 are illustrated as straight channels but the channels can be any desired configuration (i.e., diagonal with respect to the mat dimensions, herringbone, curvilinear, etc.). Any channel configuration is suitable so long as it supports draining the solution and residue away from the contact lens components place on the mat and from the mat itself.

Both the contact lens covers and the contact lens case base are placed on the first and second regions with wall height 140a formed between channels 150 so that the contact lens case components sit above the channels 150 and solution and residue can properly drain from the mat having flowed into the channels running along the surface of the base 115 of the mat 110 from the first and second regions of the mat.

It should be noted that indented or recessed areas (i.e., first and second receptacle regions 120, 130) to receive the contact lens cover(s) and contact lens case are not required. The areas can rest on channel walls 140 that are the same height as they are on the rest of the mat. The contact lens covers and case may be placed on any portion of the mat surface, recessed or not, as long as there are channels 150 thereon crossed by some form of higher support wall to allow the solution and residue (that drains from the contact lens case covers and base onto the mat) to drain from the mat. Referring to FIG. 2, the width of channels 150 may be constant or variable. Also, in other aspects, the recessed areas do not have channels formed therein but are bordered by channels. The solution and residue will flow from the recessed areas into the adjacent channels.

The perspective view in FIG. 4 illustrates mat lip 160 that forms the outlet from which saline solution and other residue (e.g., moisture) may flow out of the drainage device 100. Mat outlet 160 may be positioned, for example, adjacent a sink or other basin such that liquid draining from the mat does not drain onto expensive counter surfaces (e.g., marble, stone, etc.) that can be stained or otherwise damaged by saline and saline residue. Liquid flows to mat outlet 160 through the drainage channels 150 by operation of gravity due to the slight taper of the based on which the mat rests. The tapered base 115 therefore allows the liquid to flow through channels 150 to mat outlet 160 and exit the drainage device 100.

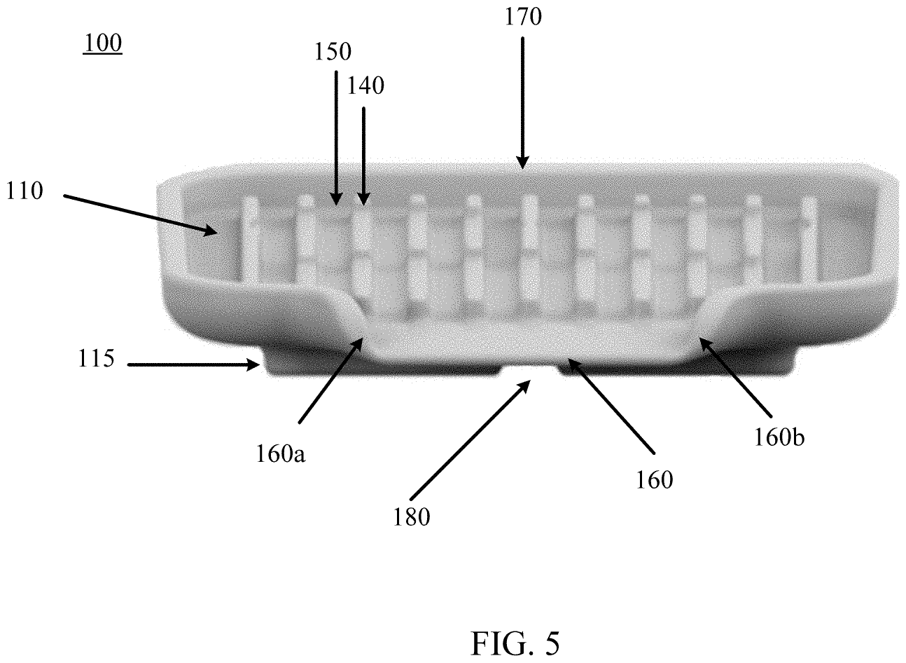

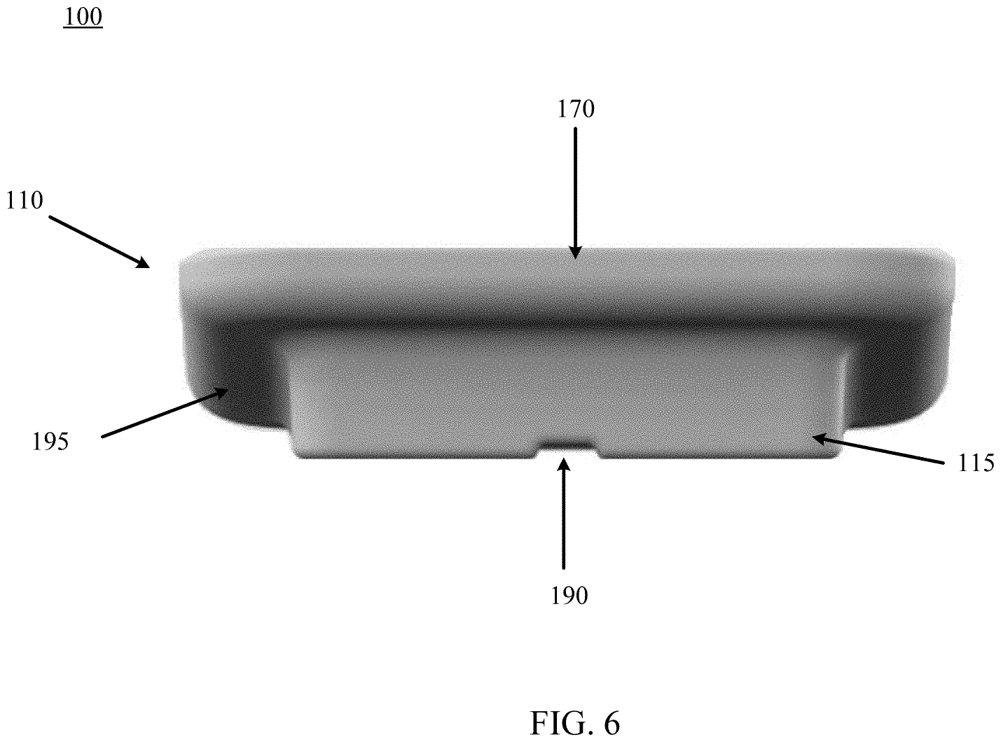

The mat 110 may further include an upper sidewall 170. Upper sidewall 170 extends partially around the perimeter of mat 110 to prevent saline solution and other residue from spilling from elsewhere other than mat outlet 160. As shown in FIG. 5, sidewall 170 extends about the portion of the perimeter of the mat 110 from one end of the outlet 160a to the other end of outlet 160b. Referring to FIG. 6, there illustrates that a channel 150 extends along the perimeter of the upper sidewall 170 to ensure that the mat is relatively free of points where the solution draining from the contact lens case components will be blocked from draining from the mat.

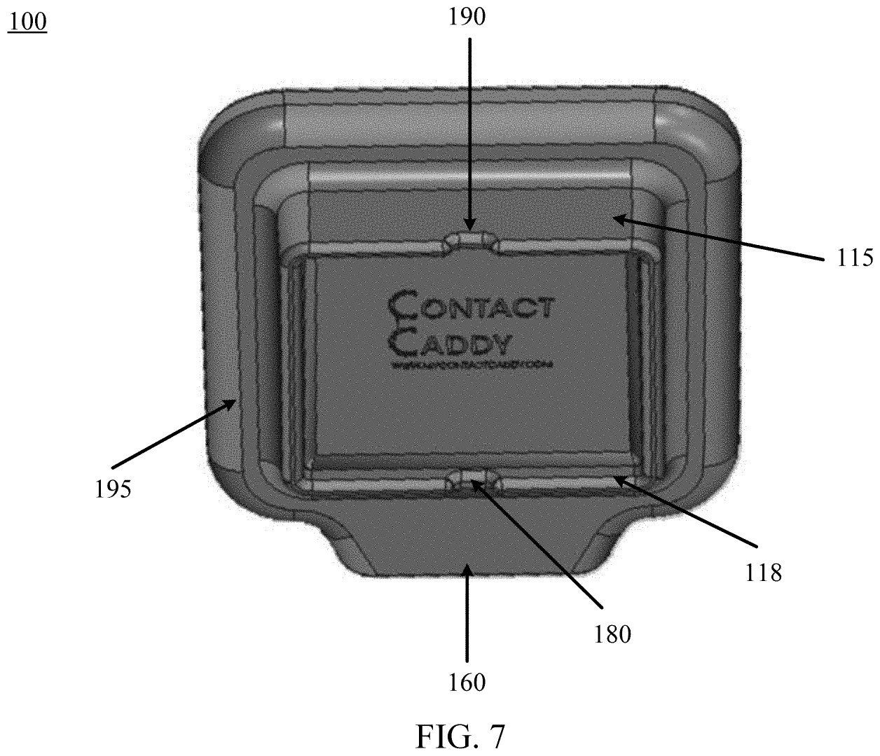

The mat 110 may also include a bottom surface 195 as shown in FIG. 6. Bottom surface 195 may extend beyond the base 115. This is further illustrated by FIG. 7. FIG. 7 shows bottom surface 195 extending past the base 115. As illustrated in FIG. 7, the perimeter of the base 115 is defined by sidewall 118 on all sides of base 115. As further illustrated in FIG. 7, the base sidewalls 118 provide a flat support for the mat 110. As illustrate, base 115 is not solid, but has the perimeter support 118 and is otherwise open at the bottom. The design and configuration of the base is largely a matter of design choice, insofar as the base supports liquid drainage from the mat. The bottom surface makes it easier to handle the mat and place it on a surface. The base sidewall is illustrated having a front notch 180 and rear notch 190 which form openings into the open base area defined by the base sidewalls 118. Such notches are an optional feature and their placement can be different from the illustrated placement. The front notch 180 and the rear notch 190 may be opposite one another and/or be aligned along a common axis. The notches may permit airflow within the open base area defined by base sidewalls 118 to facilitate drying after the mat has been washed.

From the foregoing description, one of ordinary skill in the art can easily ascertain the characteristics of the device described herein and, without departing from the spirit and scope thereof, can make various changes and modifications to the device to adapt it to various usages and conditions in accordance with the scope of the appended claims.

1. A drainage device for allowing contact lens components to dry thereon, the drainage device comprising:
a mat, the mat comprising a base with a plurality of channels formed thereon wherein the channels have spaced apart walls adapted to support a component of a contact lens case above the channels, wherein the mat is made of a non-absorbent material; and
wherein the base is sloped downward toward one end of the mat and the plurality of channels conduct liquid that drains from wet contact lens components placed thereon to the downward end of the mat to permit liquid to drain from the mat.

2. The drainage device of claim 1, wherein the mat has one or more receptacle regions thereon, the receptacle regions defining a shape that corresponds to the shape of a contact lens case component, the shaped region defined by spaced apart walls that have a height extending from the base that is less than the height of the spaced apart walls outside the one or more receptacle regions.

3. The drainage device of claim 2, wherein the mat comprises a first receptacle region and a second receptacle region, wherein the first receptacle region is dimensioned to receive one or more covers for a contact lens receptacle in a contact lens case, and wherein the second receptacle regions is dimensioned to receive a base component of the contact lens case, and further wherein a perimeter of each of the first and second receptacle regions is defined by a plurality of transitions from the height of the spaced apart walls in the first and second regions to the height of the spaced apart walls outside the first and second receptacle regions.

4. The drainage device of claim 3, wherein the spaced apart walls have a uniform wall height in the first and second receptacle regions and a uniform wall height outside the first and second receptacle regions.

5. The drainage device of claim 3, wherein the first receptacle region is shaped to receive two contact lens covers, each cover shaped to cover one contact lens receptacle in a contact lens base that comprises two contact lens receptacles.

6. The drainage device of claim 5, wherein the first and second receptacle regions are dimensioned to be slightly larger than dimensions of the contact lens component that the shaped regions is shaped to receive.

7. The drainage device of claim 1, wherein the mat is made of a flexible, non-absorbent material.

8. The drainage device of claim 7, wherein the mat is made of a synthetic material.

9. The drainage device of claim 8, wherein the mat is made of a washable material.

10. The drainage device of claim 9, wherein the mat is made of a medical grade plastic material or a medical grade silicone material.

11. The drainage device of claim 10, wherein the mat is made of a medical grade silicone material.

12. The drainage device of claim 1, further comprising:
a lip at the downward end of the mat, that allows liquid to drain from the mat.

13. The drainage device of claim 12, wherein the lip is defined by a mat sidewall that extends around the mat from a first end of the lip to a second end of the lip opposite the first end of the lip.

14. The drainage device if claim 1, wherein the base comprises a base sidewall extending about a perimeter of the base that is on an end of the base distal to the mat supported by the base.

15. The drainage device of claim 14, wherein the base sidewall surrounds an open region under the base.

16. The drainage device of claim 15, wherein the base sidewall includes at least one notch.

17. The drainage device of claim 16, wherein the at least one notch is a first notch and a second notch; and wherein the first notch and the second notch are opposite one another.

18. The drainage device of claim 1, wherein the mat has a perimeter and the perimeter of the mat is greater than the perimeter of the base.
